(19) United States
(12) Patent Application Publication
(10) Pub. No.: US 2024/0049957 A1
(43) Pub. Date: Feb. 15, 2024

(54) System And Method For Mirroring Conditions Associated With A Medical Device Reprocessor

(71) Applicant: ASP Global Manufacturing GMBH, Schaffhausen (CH)

(72) Inventor: Christopher C. Coker, Irvine, CA (US)

(73) Assignee: ASP Global Manufacturing GMBH, Schaffhausen (CH)

(21) Appl. No.: 18/267,859

(22) PCT Filed: Dec. 2, 2021
(86) PCT No.: PCT/IB2021/000854

§ 371 Date: Jun. 16, 2023

Related U.S. Application Data
(60) Provisional application No. 63/126,614, filed on Dec. 17, 2020.

(51) Int. Cl. A61B 1/12; A61L 2/18; A61L 2/28

(52) U.S. Cl. CPC A61B 1/125; A61B 1/123; A61B 1/128; A61L 2/18; A61L 2/28; A61L 2202/14; A61L 2202/24

(57) ABSTRACT

A system and method for reprocessing an internal channel of a medical device directs one or more fluids over and through the medical device. The system and method includes a module that contains an indicator. The module is located separate from the medical device that is positioned within the reprocessing system. One or more conditions within the module are maintained to mirror or substantially match the same one or more conditions within the portion of the reprocessing system containing the medical device. A decontamination assessment as to the medical device is made by evaluating the indicator within the module.

### PRIORITY

This application claims priority to U.S. Provisional Patent Application Ser. No. 63/126,614, filed Dec. 17, 2020, entitled “System and Method for Mirroring Conditions Associated with a Medical Device Reprocessor,” the disclosure of which is incorporated by reference herein.

### BACKGROUND

The below discussion relates to the reprocessing (i.e., decontamination) of endoscopes and other instruments that are used in medical procedures. In particular, the below discussion relates to an apparatus and a method that may be used to reprocess a medical device such as an endoscope after the medical device has been used in a first medical procedure, such that the medical device may be safely used in a subsequent medical procedure. While the below discussion will speak mainly in terms of an endoscope, it should be understood that the discussion may also equally apply to certain other medical devices.

An endoscope may have one or more working channels or lumens extending along at least a portion of the length of the endoscope. Such channels may be configured to provide a pathway for passage of other medical devices, etc., into an anatomical region within a patient. These channels may be difficult to clean and/or disinfect using certain primitive cleaning and/or disinfecting techniques. Thus, the endoscope may be placed in a reprocessing system that is particularly configured to clean endoscopes, including the channels within endoscopes. Such an endoscope reprocessing system may wash and disinfect the endoscope. Such an endoscope reprocessing system may include a basin that is configured to receive the endoscope, with a pump that flows cleaning fluids over the exterior of the endoscope within the basin. The system may also include ports that couple with the working channels of the endoscope and associated pumps that flow cleaning fluids through the working channels of the endoscope. The process executed by such a dedicated endoscope reprocessing system may include a detergent washing cycle, followed by a rinsing cycle, followed by a sterilization or disinfection cycle, followed by another rinsing cycle. The sterilization or disinfection cycle may employ disinfectant solution and water rinses. The final rinsing cycle concludes with purging the endoscope channels with compressed air. Optionally, the process may further include an alcohol rinsing cycle in which the endoscope channels are filled with alcohol and then purged with compressed air to facilitate drying of the channels and thereby enhancing the decontamination effects of the process.

Examples of systems and methods that may be used to reprocess a used endoscope are described in U.S. Pat. No. 6,986,736, entitled “Automated Endoscope Reprocessor Connection with Integrity Testing,” issued Jan. 17, 2006; U.S. Pat. No. 7,479,257, entitled “Automated Endoscope Reprocessor Solution Testing,” issued Jan. 20, 2009; U.S. Pat. No. 7,686,761, entitled “Method of Detecting Proper Connection of an Endoscope to an Endoscope Reprocessor,” issued Mar. 30, 2010; U.S. Pat. No. 8,246,909, entitled “Automated Endoscope Reprocessor Germicide Concentration Monitoring System and Method,” issued Aug. 21, 2012; U.S. Pat. No. 8,246,909, entitled “Automated Endoscope Reprocessor Germicide Concentration Monitoring System and Method,” issued Aug. 21, 2012; U.S. Pat. No. 10,201,269, entitled “Apparatus and Method for Reprocessing a Medical Device,” issued on Feb. 12, 2019; U.S. Pat. No. 10,702,619, entitled “Apparatus and Method to Measure Concentration of Disinfectant in Medical Device Reprocessing system,” issued on Jul. 7, 2020; U.S. Patent Pub. No. 20190076009A1, entitled “Apparatus and Method to Asynchronously Fill and Purge Channels of Endoscope Simultaneously,” published Mar. 14, 2019; U.S. Pat. No. 10,792,386, entitled “Apparatus and Method to Repeatedly Fill and Purge Channels of Endoscope,” issued on Oct. 6, 2020; and PCT Patent App. No. PCT/US20/36254, entitled “System and Method for Drying Channels of Medical Instrument During Cleaning,” filed Jun. 5, 2020, the disclosures of all the above-referenced patents, publications, and applications are incorporated by reference herein. An example of a commercially available endoscope reprocessing system is the EVOTECH® Endoscope Cleaner and Reprocessor (ECR) by Advanced Sterilization Products of Irvine, California.

While a variety of systems and methods have been made and used to reprocess medical devices, it is believed that no one prior to the inventor(s) has made or used the technology as described herein.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a front elevational view of an exemplary reprocessing system;

FIG. 2 depicts a schematic diagram of the reprocessing system of FIG. 1, with only a single decontamination basin shown for clarity;

FIG. 3 depicts a cross-sectional side view of proximal and distal portions of an endoscope that may be decontaminated using the reprocessing system of FIG. 1;

FIG. 4 a depicts a flow diagram illustrating an exemplary reprocessing method useable by the reprocessing system of FIG. 1.

FIG. 5 depicts a schematic diagram of another reprocessing system of FIG. 1, with only a single decontamination basin shown for clarity, and including an indicator module;

FIG. 6 depicts a schematic diagram of the indicator module of FIG. 5; and

FIG. 7 depicts a flow diagram illustrating an exemplary reprocessing method useable by the reprocessing system of FIG. 5.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

### I. EXEMPLARY MEDICAL DEVICE REPROCESSING APPARATUS

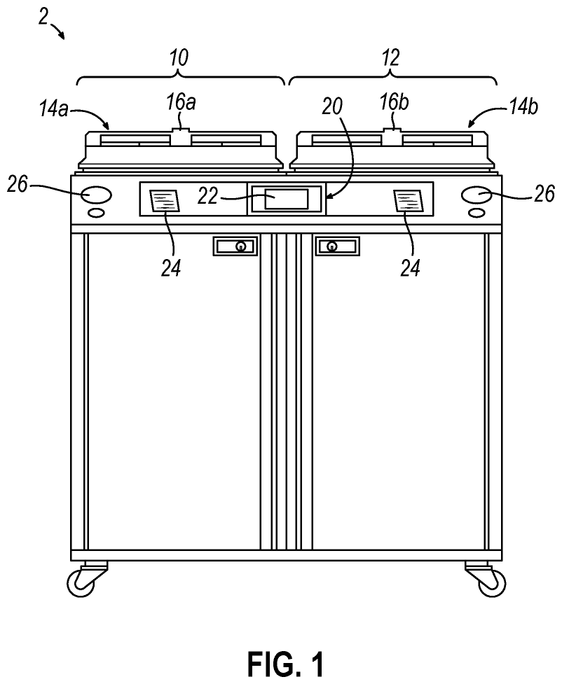

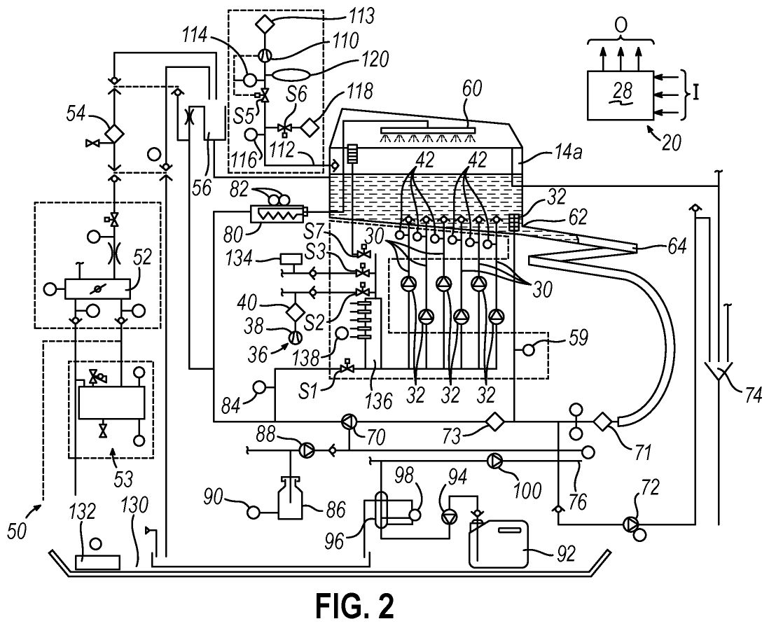

FIGS. 1-2 show an exemplary reprocessing system (2) that may be used to decontaminate endoscopes and other medical devices that include channels or lumens formed therethrough. System (2) of this example generally includes a first station (10) and a second station (12). Stations (10, 12) are at least substantially similar in all respects to provide for the decontamination of two different medical devices simultaneously or in series. First and second decontamination basins (14a, 14b) receive the contaminated devices. Each basin (14a, 14b) is selectively sealed by a respective lid (16a, 16b). In the present example, lids (16a, 16b) cooperate with respective basins (14a, 14b) to provide a microbe-blocking relationship to prevent the entrance of environmental microbes into basins (14a, 14b) during decontamination operations. By way of example only, lids (16a, 16b) may include a microbe removal or HEPA air filter formed therein for venting.

A control system (20) includes one or more microcontrollers, such as a programmable logic controller (PLC), for controlling decontamination and user interface operations. Although one control system (20) is shown herein as controlling both decontamination stations (10, 12), those skilled in the art will recognize that each station (10, 12) can include a dedicated control system. A visual display (22) displays decontamination parameters and machine conditions for an operator, and at least one printer (24) prints a hard copy output of the decontamination parameters for a record to be filed or attached to the decontaminated device or its storage packaging. It should be understood that printer (24) is merely optional. In some versions, visual display (22) is combined with a touch screen input device. In addition, or in the alternative, a keypad and/or other user input feature is provided for input of decontamination process parameters and for machine control. Other visual gauges (26) such as pressure meters and the like provide digital or analog output of decontamination or medical device leak testing data.

FIG. 2 diagrammatically illustrates just one decontamination station (10) of reprocessing system (2), but those skilled in the art will recognize that decontamination station (12) may be configured and operable just like decontamination station (10). It should also be understood that reprocessing system (2) may be provided with just one single decontamination station (10, 12) or more than two decontamination stations (10, 12).

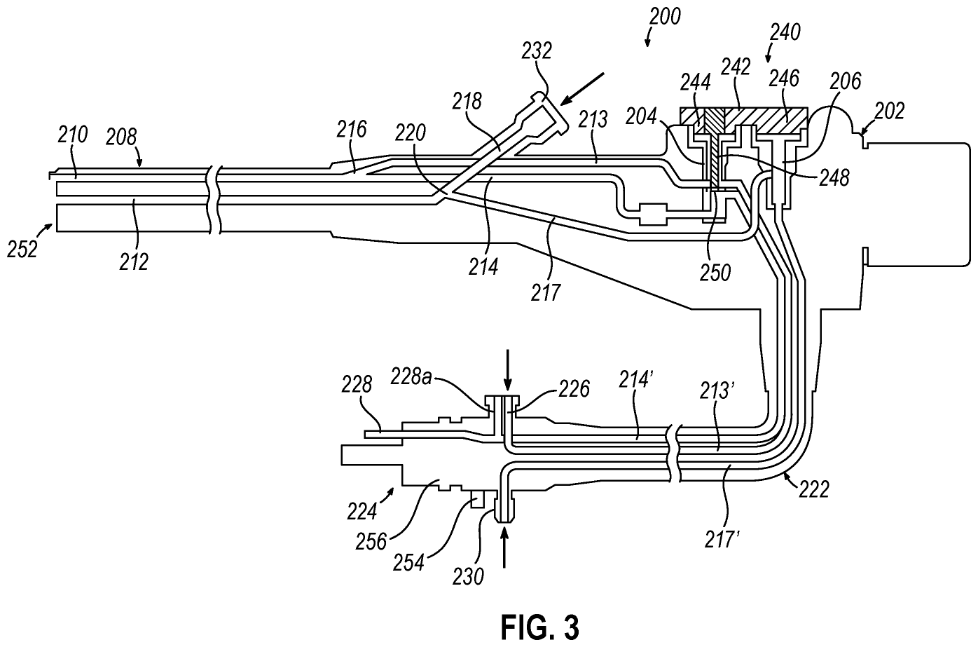

Decontamination basin (14a) receives an endoscope (200) (see FIG. 3) or other medical device therein for decontamination. Any internal channels of endoscope (200) are connected with flush conduits, such as flush lines (30). Each flush line (30) is connected to an outlet of a corresponding pump (32), such that each flush line (30) has a dedicated pump (32) in this example. Pumps (32) of the present example comprise peristaltic pumps that pump fluid, such as liquid and air, through the flush lines (30) and any internal channels of endoscope (200). Alternatively, any other suitable kind of pump(s) may be used. In the present example, pumps (32) can either draw liquid from basin (14a) through a filtered drain and a valve (S1); or draw decontaminated air from an air supply system (36) through a valve (S2). Air supply system (36) of the present example includes a pump (38) and a microbe removal air filter (40) that filters microbes from an incoming air stream.

A pressure switch or sensor (42) is in fluid communication with each flush line (30) for sensing excessive pressure in the flush line. Any excessive pressure or lack of flow sensed may be indicative of a partial or complete blockage (e.g., by bodily tissue or dried bodily fluids) in an endoscope (200) channel to which the relevant flush line (30) is connected. The isolation of each flush line (30) relative to the other flush lines (30) allows the particular blocked channel to be easily identified and isolated, depending upon which sensor (42) senses excessive pressure or lack of flow.

Basin (14a) is in fluid communication with a water source (50), such as a utility or tap water connection including hot and cold inlets, and a mixing valve (52) flowing into a break tank (56). A microbe removal filter (54), such as a 0.2 μm or smaller absolute pore size filter, decontaminates the incoming water, which is delivered into break tank (56) through the air gap to prevent backflow. A sensor (59) monitors liquid levels within basin (14a). An optional water heater (53) can be provided if an appropriate source of hot water is not available. The condition of filter (54) can be monitored by directly monitoring the flow rate of water therethrough or indirectly by monitoring the basin fill time using a float switch or the like. When the flow rate drops below a select threshold, this indicates a partially clogged filter element that requires replacement.

A basin drain (62) drains liquid from basin (14a) through an enlarged helical tube (64) into which elongated portions of endoscope (200) can be inserted. Drain (62) is in fluid communication with a recirculation pump (70) and a drain pump (72). Recirculation pump (70) recirculates liquid from basin drain (62) to a spray nozzle assembly (60), which sprays the liquid into basin (14a) and onto endoscope (200). A coarse screen (71) and a fine screen (73) filter out particles in the recirculating fluid. Drain pump (72) pumps liquid from basin drain (62) to a utility drain (74). A level sensor (76) monitors the flow of liquid from pump (72) to utility drain (74). Pumps (70, 72) can be simultaneously operated such that liquid is sprayed into basin (14a) while basin (14a) is being drained, to encourage the flow of residue out of basin (14a) and off of endoscope (200). Of course, a single pump and a valve assembly could replace dual pumps (70, 72).

An inline heater (80) with temperature sensors (82), upstream of recirculation pump (70), heats the liquid to optimum temperatures for cleaning and/or disinfection. A pressure switch or sensor (84) measures pressure downstream of circulation pump (70). In some variations, a flow sensor is used instead of pressure sensor (84), to measure fluid flow downstream of circulation pump (70). Detergent solution (86) is metered into the flow downstream of circulation pump (70) via a metering pump (88). A float switch (90) indicates the level of detergent (86) available. Disinfectant (92) is metered into the flow upstream of circulation pump (70) via a metering pump (94). To more accurately meter disinfectant (92), pump (94) fills a metering pre-chamber (96) under control of a fluid level switch (98) and control system (20). By way of example only, disinfectant solution (92) may comprise an activated glutaraldehyde salutation, such as CIDEX® Activated Glutaraldehyde Solution by Advanced Sterilization Products of Irvine, California. By way of further example only, disinfectant solution (92) may comprise ortho-phthalaldehyde (OPA), such as CIDEX® ortho-phthalaldeyde solution by Advanced Sterilization Products of Irvine, California. By way of further example only, disinfectant solution (92) may comprise peracetic acid (PAA).

Some endoscopes (200) include a flexible outer housing or sheath surrounding the individual tubular members and the like that form the interior channels and other parts of endoscope (200). This housing defines a closed interior space, which is isolated from patient tissues and fluids during medical procedures. It may be important that the sheath be maintained intact, without cuts or other holes that would allow contamination of the interior space beneath the sheath. Therefore, reprocessing system (2) of the present example includes means for testing the integrity of such a sheath. In particular, an air pump (e.g., pump (38) or another pump (110)) pressurizes the interior space defined by the sheath of endoscope (200) through a conduit (112) and a valve (S5). In the present example, a HEPA or other microbe-removing filter (113) removes microbes from the pressurizing air. A pressure regulator (114) prevents accidental over pressurization of the sheath. Upon full pressurization, valve (S5) is closed and a pressure sensor (116) looks for a drop in pressure in conduit (112), which would indicate the escape of air through the sheath of endoscope (200). A valve (S6) selectively vents conduit (112) and the sheath of endoscope (200) through an optional filter (118) when the testing procedure is complete. An air buffer (120) smoothes out pulsation of pressure from air pump (110).

In the present example, each station (10, 12) also contains a drip basin (130) and spill sensor (132) to alert the operator to potential leaks. Also, an alcohol supply (134), controlled by a valve (S3), can supply alcohol to channel pumps (32) after rinsing steps, to assist in removing water from channels (210, 212, 213, 214, 217, 218) of endoscope (200).

Flow rates in lines (30) can be monitored via channel pumps (32) and pressure sensors (42). If one of pressure sensors (42) detects too high a pressure, the associated pump (32) is deactivated. The flow rate of pump (32) and its activated duration time provide a reasonable indication of the flow rate in an associated line (30). These flow rates are monitored during the process to check for blockages in any of the channels of endoscope (200). Alternatively, the decay in the pressure from the time pump (32) cycles off can also be used to estimate the flow rate, with faster decay rates being associated with higher flow rates.

A more accurate measurement of flow rate in an individual channel may be desirable to detect subtler blockages. To that end, a metering tube (136) having a plurality of level indicating sensors (138) fluidly connects to the inputs of channel pumps (32). In some versions, a reference connection is provided at a low point in metering tube (136) and a plurality of sensors (138) are arranged vertically above the reference connection. By passing a current from the reference point through the fluid to sensors (138), it can be determined which sensors (138) are immersed and therefore determine the level within metering tube (136). In addition, or in the alternative, any other suitable components and techniques may be used to sense fluid levels. By shutting valve (S1) and opening a vent valve (S7), channel pumps (32) draw exclusively from metering tube (136). The amount of fluid being drawn can be very accurately determined based upon sensors (138). By running each channel pump (32) in isolation, the flow therethrough can be accurately determined based upon the time and the volume of fluid emptied from metering tube (136).

In addition to the input and output devices described above, all of the electrical and electromechanical devices shown are operatively connected to and controlled by control system (20). Specifically, and without limitation, switches and sensors (42, 59, 76, 84, 90, 98, 114, 116, 132 136) provide input (I) to microcontroller (28), which controls the cleaning and/or disinfection cycles and other machine operations in accordance therewith. For example, microcontroller (28) includes outputs (O) that are operatively connected to pumps (32, 38, 70, 72, 88, 94, 100, 110), valves (S1, S2, S3, S5, S6, S7), and heater (80) to control these devices for effective cleaning and/or disinfection cycles and other operations.

### II. EXEMPLARY MEDICAL DEVICE FOR USE WITH REPROCESSING APPARATUS

As shown in FIG. 3, endoscope (200) has a head part (202). Head part (202) includes openings (204, 206) formed therein. During normal use of endoscope (200), an air/water valve (not shown) and a suction valve (not shown) are arranged in openings (204, 206). A flexible shaft (208) is attached to head part (202). A combined air/water channel (210) and a combined suction/biopsy channel (212) are accommodated in shaft (208). A separate air channel (213) and water channel (214) are also arranged in head part (202) and merge into air/water channel (210) at the location of a joining point (216). It will be appreciated that the term “joining point” as used herein refers to an intersecting junction rather than being limited to a geometrical point and, the terms may be used interchangeably. Furthermore, a separate suction channel (217) and biopsy channel (218) are accommodated in head part (202) and merge into suction/biopsy channel (212) at the location of a joining point (220).

In head part (202), air channel (213) and water channel (214) open into opening (204) for the air/water valve (not shown). Suction channel (217) opens into opening (206) for the suction valve (not shown). Furthermore, a flexible feed hose (222) connects to head part (202) and accommodates channels (213′, 214′, 217′), which are connected to air channel (213), water channel (214), and suction channel (217) via respective openings (204, 206). In practice, feed hose (222) may also be referred to as the light-conductor casing. The mutually connecting air channels (213, 213′) will collectively be referred to below as air channel (213). The mutually connecting water channels (214, 214′) will collectively be referred to below as water channel (214). The mutually connecting suction channels (217, 217′) will collectively be referred to below as suction channel (217). A connection (226) for air channel (213), connections (228, 228a) for water channel (214), and a connection (230) for suction channel (217) are arranged on the end section (224) (also referred to as the light conductor connector) of flexible hose (222). When the connection (226) is in use, connection (228a) is closed off. A connection (232) for biopsy channel (218) is arranged on head part (202).

A channel separator (240) is shown inserted into openings (204, 206). Channel separator (240) comprises a body (242) and plug members (244, 246), which occlude respective openings (204, 206). A coaxial insert (248) on plug member (244) extends inwardly of opening (204) and terminates in an annular flange (250), which occludes a portion of opening (204) to separate channel (213) from channel (214). By connecting lines (30) to openings (226, 228, 228a, 230, 232), liquid for cleaning and disinfection can be flowed through endoscope channels (213, 214, 217, 218) and out of a distal tip (252) of endoscope (200) via channels (210, 212). Channel separator (240) ensures that such liquid flows all the way through endoscope (200) without leaking out of openings (204, 206); and isolates channels (213, 214) from each other so that each channel (213, 214) has its own independent flow path. One of skill in the art will appreciate that various endoscopes having differing arrangements of channels and openings may require modifications to channel separator (240) to accommodate such differences while occluding ports in head (202) and keeping channels separated from each other so that each channel can be flushed independently of the other channels. Otherwise, a blockage in one channel might merely redirect flow to a connected unblocked channel.

A leakage port (254) on end section (224) leads into an interior portion (256) of endoscope (200) and is used to check for the physical integrity thereof, namely to ensure that no leakage has formed between any of the channels and the interior (256) or from the exterior to the interior (256).

### III. EXEMPLARY MEDICAL DEVICE REPROCESSING METHOD

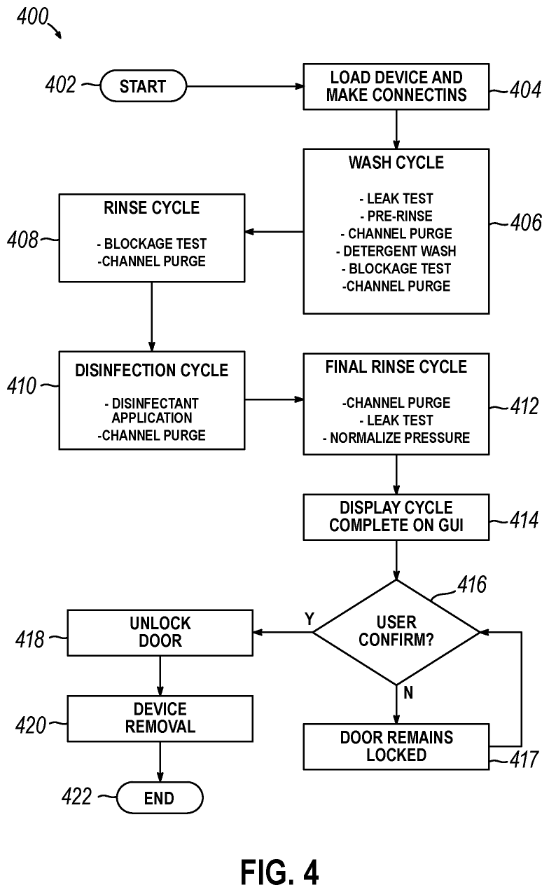

Referring to FIG. 4, in an exemplary use of reprocessing system (2), an operator may start by loading a device for reprocessing and making any needed connections with the device (404). To do so, the operator may actuate a foot pedal (not shown) to open basin lid (16a). Each lid (16a, 16b) may have its own foot pedal. In some versions, once pressure is removed from the foot pedal, the motion of lid (16a, 16b) stops. With lid (16a) open, the operator inserts shaft (208) of endoscope (200) into helical circulation tube (64). End section (224) and head section (202) of endoscope (200) are situated within basin (14a), with feed hose (222) coiled within basin (14a) with as wide a diameter as possible. Next, flush lines (30) are attached to respective endoscope openings (226, 228, 228a, 230, 232). Air line (112) is also connected to connector (254). In some versions, flush lines (30) are color coded, and guide located on station (10) provides a reference for the color-coded connections.

Depending on the customer-selectable configuration, control system (20) may prompt the operator to enter a user code, patient ID, endoscope code, and/or specialist code. This information may be entered manually (e.g., through touch screen (22)), automatically (e.g., by using an attached barcode wand), or in any other suitable fashion. With the information entered (if required), the operator may then close lid (16a). In some versions, closing lid (16a) requires the operator to press a hardware button and a touch-screen (22) button simultaneously to provide a fail-safe mechanism for preventing the operator's hands from being caught or pinched by the closing basin lid (16a). If either the hardware button or software button is released while lid (16a) is in the process of closing, the motion of lid (16a) stops.

Once lid (16a) is closed, the operator presses a button on touch-screen (22) to begin the wash cycle (406). At the start of wash cycle (406), air pump (38) is activated and pressure within the body of endoscope (200) is monitored. When pressure reaches a predetermined level (e.g., 250 mbar), pump (38) is deactivated, and the pressure is allowed to stabilize for a certain stabilization period (e.g., 6 seconds). If pressure has not reached a certain pressure (e.g., 250 mbar) in a certain time period (e.g., 45 seconds), the program is stopped and the operator is notified of a leak. If pressure drops below a threshold (e.g., less than 100 mbar) during the stabilization period, the program is stopped and the operator is notified of the condition. Once the pressure has stabilized, the pressure drop is monitored over the course of a certain duration (e.g., 60 seconds). If the pressure drop is faster than a predetermined rate (e.g., more than 10 mbar within 60 seconds), the program is stopped and the operator is notified of the condition. If the pressure drop is slower than a predetermined rate (e.g., less than 10 mbar in 60 seconds), reprocessing system (2) continues with the next step. A slight positive pressure is held within the body of endoscope (200) during the rest of the process to prevent fluids from leaking in.

A second leak test checks the adequacy of connection to the various ports (226, 228, 228a, 230, 232) and the proper placement of channel separator (240). A quantity of water is admitted to basin (14a) to submerge the distal end of endoscope (200) in helical tube (64). Valve (S1) is closed and valve (S7) opened; and pumps (32) are run in reverse to draw a vacuum and to ultimately draw liquid into endoscope channels (210, 212). Pressure sensors (42) are monitored to make sure that the pressure in any one channel (210, 212) does not drop and/or raise by more than a predetermined amount in a given time frame. If it does, it likely indicates that one of the connections was not made correctly and air is leaking into channel (210, 212). In any event, in the presence of an unacceptable pressure drop, control system (20) will cancel the cycle and indicate a likely faulty connection, preferably with an indication of which channel (210, 212) failed.

In the event that the leak tests are passed, reprocessing system (2) continues wash cycle (406) with a pre-rinse cycle. The purpose of this step is to flush water through channels (210, 212, 213, 214, 217, 218) to remove waste material prior to washing and disinfecting endoscope (200). To initiate the pre-rinse cycle, basin (14a) is filled with filtered water and the water level is detected by pressure sensor (59) below basin (14a). The water is pumped via pumps (32) through the interior of channels (210, 212, 213, 214, 217, 218), directly to drain (74). This water is not recirculated around the exterior surfaces of endoscope (200) during this stage. As the water is being pumped through channels (210, 212, 213, 214, 217, 218), drain pump (72) is activated to ensure that basin (14a) is also emptied. Drain pump (72) will be turned off when drain switch (76) detects that the drain process is complete. During the draining process, a channel purge is executed where sterile air is blown via air pump (38) through all endoscope channels (210, 212, 213, 214, 217, 218) simultaneously, to minimize potential carryover.

Once the pre-rinse cycle is complete, wash cycle (406) continues by filling basin (14a) with warm water (e.g., approximately 35° C.). Water temperature is controlled by controlling the mix of heated and unheated water. The water level is detected by pressure sensor (59). Reprocessing system (2) then adds enzymatic detergent to the water circulating in reprocessing system (2) by means of peristaltic metering pump (88). The volume is controlled by controlling the delivery time, pump speed, and inner diameter of the tubing of pump (88). Detergent solution (86) is actively pumped throughout the internal endoscope channels (210, 212, 213, 214, 217, 218) and over the outer surface of endoscope (200) for a predetermined time period (e.g., from one to five minutes, or more particularly about three minutes), by channel pumps (32) and external circulation pump (70). Inline heater (80) keeps the temperature at a predetermined temperature (e.g., approximately about 35° C.).

After detergent solution (86) has been circulating for a certain period of time (e.g., a couple of minutes), the flow rate through channels (210, 212, 213, 214, 217, 218) is measured. If the flow rate through any channel (210, 212, 213, 214, 217, 218) is less than a predetermined rate for that channel (210, 212, 213, 214, 217, 218), the channel (210, 212, 213, 214, 217, 218) is identified as blocked, the program is stopped, and the operator is notified of the condition. Peristaltic pumps (32) are run at their predetermined flow rates and cycle off in the presence of unacceptably high pressure readings at the associated pressure sensor (42). If a channel (210, 212, 213, 214, 217, 218) is blocked, the predetermined flow rate will trigger pressure sensor (42), indicating the inability to adequately pass this flow rate. As pumps (32) are peristaltic in the present example, their operating flow rate combined with the percentage of time they are cycled off due to pressure will provide the actual flow rate. The flow rate can also be estimated based upon the decay of the pressure from the time pump (32) cycles off.

At the end of wash cycle (406), drain pump (72) is activated to remove detergent solution (86) from basin (14a) and channels (210, 212, 213, 214, 217, 218). Drain pump (72) turns off when drain level sensor (76) indicates that drainage is complete. During the drain process, sterile air is blown through all channels (210, 212, 213, 214, 217, 218) of endoscope (200) simultaneously to purge the channels and minimize potential carryover.

After wash cycle (406) is complete, reprocessing system (2) begins a rinse cycle (408). To initiate rinse cycle (408), basin (14a) is again filled with warm water (e.g., at approximately 35° C.). Water temperature is controlled by controlling the mix of heated and unheated water. The water level is detected by pressure sensor (59). The rinse water is circulated within channels (210, 212, 213, 214, 217, 218) of endoscope (200) via channel pumps (32); and over the exterior of endoscope (200) via circulation pump (70) and sprinkler arm (60) for a certain period of time (e.g., one minute). As rinse water is pumped through channels (210, 212, 213, 214, 217, 218), the flow rate through channels (210, 212, 213, 214, 217, 218) is measured and if it falls below the predetermined rate for any given channel (210, 212, 213, 214, 217, 218), that channel (210, 212, 213, 214, 217, 218) is identified as blocked, the program is stopped, and the operator is notified of the condition.

At the end of rinse cycle (408), drain pump (72) is activated to remove the rinse water from basin (14a) and channels (210, 212, 213, 214, 217, 218). Drain pump (72) turns off when drain level sensor (76) indicates that drainage is complete. During the drain process, sterile air is blown through all channels (210, 212, 213, 214, 217, 218) of endoscope (200) simultaneously to purge the channels and minimize potential carryover. In some versions, the above-described rinsing and draining cycles are repeated at least once again, to ensure maximum rinsing of detergent solution (86) from the surfaces of endoscope (200) and basin (14a).

After reprocessing system (2) has completed the desired number of rinsing and draining cycles, reprocessing system (2) proceeds to a disinfection cycle (410). To initiate disinfection cycle (410), basin (14a) is filled with very warm water (e.g., at approximately 53° C.). Water temperature is controlled by controlling the mix of heated and unheated water. The water level is detected by pressure sensor (59). During the filling process, channel pumps (32) are off in order to ensure that the disinfectant solution (92) in basin (14a) is at the in-use concentration prior to circulating through channels (210, 212, 213, 214, 217, 218) of endoscope (200).

Next, a measured volume of disinfectant solution (92) is drawn from disinfectant metering pre-chamber (96) and delivered into the water in basin (14a) via metering pump (100). The volume of disinfectant solution (92) is controlled by the positioning of fill level switch (98) relative to the bottom of metering pre-chamber (96). Metering pre-chamber (96) is filled until fill level switch (98) detects liquid. Disinfectant solution (92) is drawn from metering pre-chamber (96) until the level of disinfectant solution (92) in metering pre-chamber (96) is just below the tip of metering pre-chamber (96). After the necessary volume is dispensed, metering pre-chamber (96) is refilled from the bottle of disinfectant solution (92). Disinfectant solution (92) is not added until basin (14a) is filled, so that in case of a water supply problem, concentrated disinfectant is not left on endoscope (200) with no water to rinse it. While disinfectant solution (92) is being added, channel pumps (32) are off in order to ensure that disinfectant solution (92) in basin (14a) is at the desired in-use concentration prior to circulating through channels (210, 212, 213, 214, 217, 218) of endoscope (200).

The in-use disinfectant solution (92) is actively pumped throughout internal channels (210, 212, 213, 214, 217, 218) by pumps (32) and over the outer surface of endoscope (200) by circulation pump (70). This may be done for any suitable duration (e.g., at least 5 minutes). The temperature of the disinfectant solution (92) may be controlled by in-line heater (80) to stay at a consistent temperature (e.g., about 52.5° C.). During the disinfection process, flow through each channel (210, 212, 213, 214, 217, 218) of endoscope (200) is verified by timing the delivery of a measured quantity of solution through channel (210, 212, 213, 214, 217, 218). Valve (S1) is closed, and valve (S7) opened, and in turn each channel pump (32) delivers a predetermined volume to its associated channel (210, 212, 213, 214, 217, 218) from metering tube (136). This volume and the time it takes to deliver the volume, provides a very accurate flow rate through the channel (210, 212, 213, 214, 217, 218). Anomalies in the flow rate from what is expected for a channel (210, 212, 213, 214, 217, 218) of that diameter and length are flagged by control system (20) and the process stopped. As in-use disinfectant solution (92) is pumped through channels (210, 212, 213, 214, 217, 218), the flow rate through channels (210, 212, 213, 214, 217, 218) is also measured as described above.

At the end of disinfection cycle (410), drain pump (72) is activated to remove disinfectant solution (92) solution from basin (14a) and channels (210, 212, 213, 214, 217, 218). During the draining process, sterile air is blown through all channels (210, 212, 213, 214, 217, 218) of endoscope (200) simultaneously to purge channels and minimize potential carryover.

After disinfectant solution (92) has been drained from basin (14a), reprocessing system (2) begins a final rinse cycle (412). To initiate cycle (412), basin (14a) is filled with sterile warm water (e.g., at approximately 45° C.) that has been passed through a filter (e.g., a 0.2 μm filter). The rinse water is circulated within channels (210, 212, 213, 214, 217, 218) by pumps (32); and over the exterior of endoscope (200) via circulation pump (70) and sprinkler arm 60) for a suitable duration (e.g., 1 minute). As rinse water is pumped through channels (210, 212, 213, 214, 217, 218), the flow rate through channels (210, 212, 213, 214, 217, 218) is measured as described above. Drain pump (72) is activated to remove the rinse water from basin (14a) and channels (210, 212, 213, 214, 217, 218). During the draining process, sterile air is blown through all channels (210, 212, 213, 214, 217, 218) of endoscope (200) simultaneously to purge channels and minimize potential carryover. In some versions, the above-described rinsing and draining cycles are repeated at least two more times, to ensure maximum rinsing of disinfectant solution (92) residuals from the surfaces of endoscope (200) and basin (14a).

After the final rinse cycle (412) is complete, reprocessing system (2) begins a final leak test. In particular, reprocessing system (2) pressurizes the body of endoscope (200) and measures the leak rate as described above. If the final leak test is successful, reprocessing system (2) indicates the successful completion of the cycles by displaying a cycle complete indication (414) on a graphical user interface (GUI), e.g., via touch-screen (22). From the time of program completion to the time at which lid (16a) is opened, pressure within the body of endoscope (200) is normalized to atmospheric pressure by opening vent valve (S5) at a predetermined rate (e.g., valve (S5) opened for 10 seconds every minute).

Depending on customer-selected configuration, reprocessing system (2) may prevent lid (16a) from being opened until receiving user confirmation (416), e.g., a valid user identification code may be entered to provide user confirmation. Information about the completed program, including the user ID, endoscope ID, specialist ID, and patient ID are stored along with the sensor data obtained throughout the program. If a printer is connected to reprocessing system (2), and if requested by the operator, a record of the disinfection program will be printed. Once user confirmation (416) has been received, lid (16a) may be unlocked (418) and opened (e.g., using the foot pedal as described above). Endoscope (200) is then removed in a device removal step (420) where endoscope (200) is disconnected from flush lines (30) and removed from basin (14a). Lid (16a) can then be closed using both the hardware and software buttons as described above. With device (200) removed from reprocessing system (2), reprocessing method (400) ends (422) and may be repeated thereafter for reprocessing additional devices, i.e. endoscopes (200).

### IV. EXEMPLARY MEDICAL DEVICE REPROCESSING APPARATUS WITH INDICATOR MODULE

With some reprocessing systems, an indicator can be used to determine when the medical device has been decontaminated. For instance, in some systems an indicator comprising a soiled test strip (also referred to as a coupon or test sample) is located in basin (14a) along with the medical device. In this manner the indicator is exposed to the same or similar reprocessing conditions as the medical device such that when the indicator shows removal of the soiled portion or area, a correlation can be made to suggest that the medical device is decontaminated. In some instances, the soil used with the indicator can be configured to represent the soil found within one or more of internal channels (210, 212, 213, 214, 217, 218) of endoscope (200).

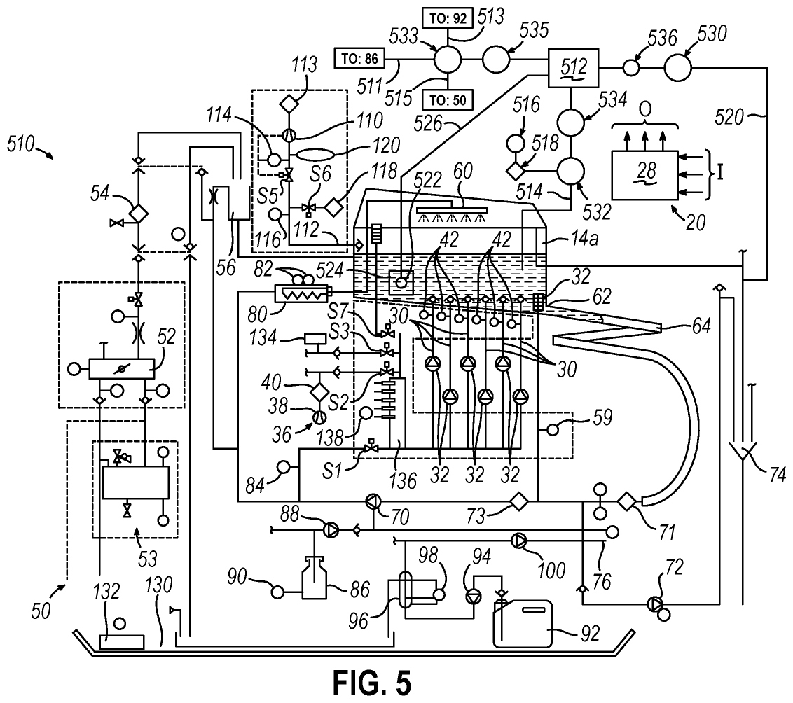

While some systems use indicators within basin (14a), in other examples it can be beneficial to use one or more indicators separate from or outside of basin (14a). FIG. 5 illustrates another exemplary decontamination station (510) usable with reprocessing system (2). Decontamination station (510) is similar to decontamination station (10) of FIG. 2 and as described above. Therefore, it should be understood that the description and details of decontamination station (10) described above with reference to FIG. 2 apply equally to decontamination station (510) described further below with respect to FIG. 5; however, decontamination station (510) includes additional features as described further below.

Decontamination station (510) includes an indicator module (512) where indicator module (512) is located outside or separate from basin (14a). However, as shown in the example of FIG. 5, indicator module (512) is fluidly connected with basin (14a) by flush line (514) such that fluids from basin (14a) can be delivered to indicator module (512). In this manner, a pump (530) is configured to draw fluid from basin (14a) to a three-way valve (532). Valve (532) is configurable to direct the fluid to a two-way isolation valve (534), which is configurable to direct the fluid into indicator module (512). With the fluid within indicator module (512), indicator module (512) has or is configured to have a comparable environment to basin (14a). In other words, indicator module (512) mirrors the environment or conditions within basin (14a).

Also fluidly connected with indicator module (512) is an air source (516). Air source (516) provides decontaminated air or sterilized air to indicator module (512). For instance, the air from air source (516) passes through a microbial filter (518) to decontaminate the air before sending along to three-way valve (532). From valve (532) the air is directed to two-way isolation valve (534), which is configurable to direct the air into indicator module (512).

In some versions, indicator module (512) is fluidly connected with a container or supply of detergent solution (86) and/or disinfectant solution (92) by flush lines (511, 513) such that detergent solution (86) and/or disinfectant solution (92) can be delivered to indicator module (512). For instance, pump (530) can be configured to draw detergent solution (86) and/or disinfectant solution (92) from their respective containers or supply sources to a valve (533). Valve (533) is configurable to direct the detergent solution (86) and/or disinfectant solution (92) to a two-way isolation valve (535), which is configurable to direct the detergent solution (86) and/or disinfectant solution (92) into indicator module (512).

Directing detergent solution (86) and/or disinfectant solution (92) directly from their respective sources to indicator module (512) allows for delivering a desired concentration of either of these fluids to indicator module (512). This can be desirable when the conditions within basin (14a) may differ from the conditions within the one or more channels of the device, e.g. endoscope (200), and indicator module (512) is meant to mirror the conditions within the one or more channels. For instance, in some reprocessing methods, higher concentrations of detergent solution (86) and/or disinfectant solution (92) may be directly delivered to the one or more internal channels of endoscope (200), while lower concentrations of these fluids may be held within basin (14a) and contacting the exterior of endoscope (200). These fluids directed to the one or more internal channels of endoscope (200) may remain within the one or more channels for a prescribed dwell time before being flushed from the respective channels.

As will be described further below, when using an indicator within indicator module (512) to evaluate decontamination status or progress, it is desirable in at least some instances for the indicator to be subject to the same or similar conditions as those experienced within the one or more internal channels of the device. While the above example provides for scenarios where differing fluid concentrations may be used within the one or more internal channels of endoscope (200) and basin (14a), in other versions, the fluid within basin (14a) is identical with the fluid that circulates through the one or more internal channels of endoscope (200). In some examples of such versions the fluid from basin (14a) is pumped through the one or more channels such that the fluids are the same. In such examples, fluid from basin (14a) is also directable to indicator module (512) as described above.

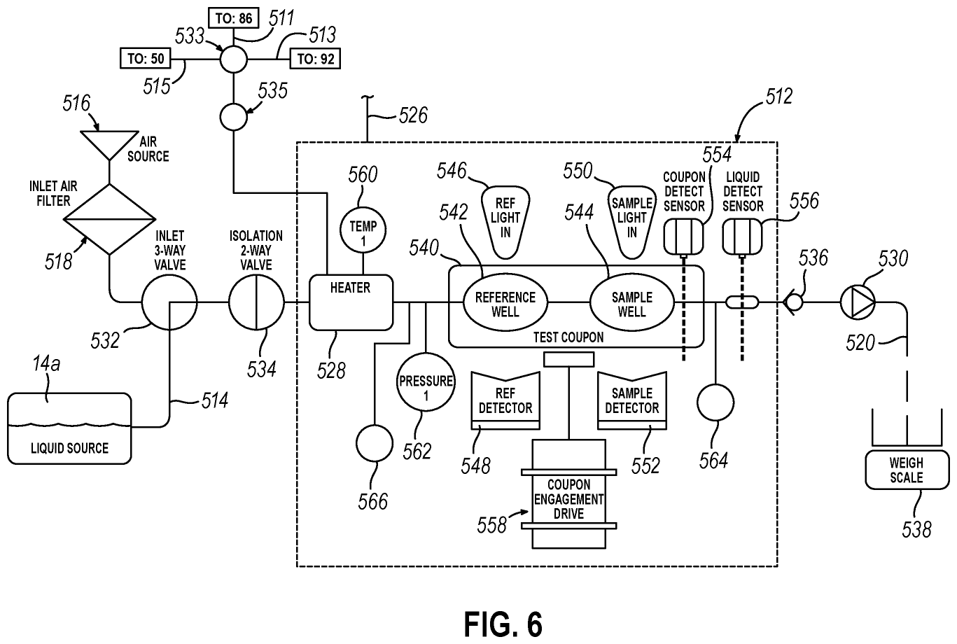

Indicator module (512) further is fluidly connected with drain (74) by drain line (520). In the present example, fluid is directed to drain (74) rather than being recirculated to avoid the risk of decontamination. A check valve (536) is located downstream of indicator module (512) before pump (530) and drain (74). With check valve (536) in an open position or state, fluid can be directed to drain (74) either by gravity or with the assistance of pump (530). In some versions as shown in FIG. 6, a scale (538) is used to measure the weight of the fluid directed to the drain. With this information, the amount of fluid passing through indicator module (512) can be calculated based on the weight and known density of the fluid.

As also shown in FIG. 5, decontamination station (510) includes a temperature probe (522) positioned within basin (14a). Surrounding temperature probe (522) is an optional material (524) that is configured to simulate the thermal properties of endoscope (200). In this manner, material (524) works in conjunction with temperature probe (522) to represent or mirror the temperature of the fluid that is delivered to internal channels (210, 212, 213, 214, 217, 218) of endoscope (200). Temperature probe (522) may be located within basin (14a) in a location where the temperature of the fluid within basin (14a) is known to match or substantially match the temperature of the fluid delivered to internal channels (210, 212, 213, 214, 217, 218). In another example, temperature probe (522) is positioned within basin (14a) at a location where the temperature of the fluid is known to be the least optimal. In this way, the temperature of the fluid within channels (210, 212, 213, 214, 217, 218) of endoscope (200) can be assumed to be more optimal than the measured temperature detected by temperature probe (522) in basin (14a). In this example, a higher temperature is considered more optimal as it promotes better cleaning and disinfection results. In other examples, the more optimal temperature can be the temperature that is closest to a setpoint temperature.

Temperature probe (522) is in communication with indicator module (512) by way of wire or cable (526). Wire (526) communicates temperature information or data from temperature probe (522) to a temperature control feature (528) of module (512). In some instances, wire (526) instead, or in addition, communicates temperature information or data from temperature probe (522) to controller (20), which then communicates with temperature control feature (528) as described further below. Temperature control feature (528) is operable to provide heating or cooling to the fluid directed to indicator module (512) as may be needed or desired such that the fluid temperature within indicator module (512) mirrors or matches that in basin (14a). As shown in FIG. 6, temperature control feature (528) is located within indicator module (512) and is thus separate from basin (14a) where endoscope (200) is placed. In some versions temperature control feature (528) comprises a resistive heater such as an adhesive backed thin film heater. Based on the temperature data from probe (522), a temperature setpoint associated with temperature control feature (528) can be set such that the temperature of the fluid within module (512) matches or substantially matches the temperature of the fluid within basin (14a). By way of example only, temperature control feature (528) may be used to heat the fluid within module (512) due to heat loss experienced when fluid is transferred from basin (14a) to module (512). In other instances, temperature control feature (528) may be used to heat or cool the fluid within module (512) based on temperature changes that may occur within basin (14a) based on reprocessing procedures or steps. With this configuration, the setpoint of temperature control feature (528) can be changed dynamically based on the conditions experienced within basin (14a). In some versions, the temperature control associated with the fluid in indicator module (512) is configured to respond faster than the expected rate of temperature change experienced in basin (14a).

With the configuration of decontamination station (510) as described above, an indicator such as a test strip or test coupon can be placed remote from or external to basin (14a) that contains endoscope (200) and yet still retain the ability to respond in a similar manner as if the test strip or test coupon were located inside basin (14a) with endscope (200). In a more specific example referring to FIG. 6, indicator module (512) comprises an indicator configured as a test strip or test coupon (540), which contains a reference well (542) and a sample well (544). Additionally, a reference light source (546) and a reference light detector (548) are positioned adjacent reference well (542). In this manner, light from reference light source (546) passes through reference well (542) and is detected by reference light detector (548). Similarly, a sample light source (550) and a sample light detector (552) are positioned adjacent sample well (544). In this manner, light from sample light source (550) passes through sample well (544) and is detected by sample light detector (552).

Reference well (542) is used to establish a known clean condition. For instance, reference well (542) may be a pre-cleaned surface on test coupon (540). Accordingly, the amount of light transmitted through reference well (542) and detected by reference light detector (548) represents a defined clean state or a target light transmission for when a clean state has been achieved. Sample well (544) is configured to contain a material sample that is representative of the soil that is contained within the channels of endoscope (200). In some instances, sample well (544) has a material sample that represents a worst-case soil that would be considered more difficult to clean or remove than even the soil contained within endoscope (200). In either approach, when test coupon (540) has been sufficiently exposed to fluid from basin (14a) that has been transferred to indicator module (512), the contents of sample well (544) will become clean. This will be evidenced by the light transmission through sample well (544) and detected by sample light detector (552) matching or substantially matching the detected light transmission through reference well (542) and detected by reference light detector (548).

Indicator module (512), in the present example, also includes a coupon detection sensor (554), a liquid detection sensor (556), and a test coupon loading feature (558). Test coupon loading feature (558) is configured to load test coupon (540) into position relative to reference light source (546), sample light source (550), reference light detector (548), and sample light detector (552). Coupon detection sensor (554) is configured to verify that test coupon (540) is loaded properly such that light from light sources (546, 550) will be transmitted through respective reference well (542) and sample well (544) for detection by reference light detector (548) and sample light detector (552). Liquid detection sensor (556) confirms that fluid from basin (14a) is contained within indicator module (512) such that test coupon (540) and its wells (542, 544) are exposed to the fluid.

As mentioned above, the temperature within indicator module (512) is dynamically controlled to match or substantially match that of basin (14a). By way of example, the temperature from probe (522) can be provided as an input (I) to control system (20), which is configurable to control temperature control feature (528) based on this temperature input to maintain the temperature of fluid within indicator module (512) to match the temperature of fluid within basin (14a). In some versions, indicator module (512) includes a temperature sensor (560) that determines the temperature within indicator module (512). The temperature from sensor (560) can also be provided to control system (20) as an input (I), and in this manner there is a temperature feedback loop where the temperature within indicator module (512) can be directly compared with the temperature reading provided by probe (522) in basin (14a). Based on these inputs control system (20) is configured to control temperature control feature (528) and in such an example control system (20) can be configured as a PID controller. It should further be understood that temperature probe (522) can also be used to control the endoscope reprocessor system or decontamination station (510), such as verifying temperature parameters are met or satisfied within basin (14a) before advancing to the next reprocessing stage.

Indicator module (512), in some versions, uses other parameters that are detected or measured by various sensors to further mirror the conditions within indicator module (512) with those within basin (14a). For example, indicator module (512) in some versions includes a pressure sensor (562) configured to detect the pressure within indicator module (512). In this manner, the pressure that sample well (544) is subject to is known or determinable. In addition to temperature, pressure can also impact cleaning ability and time to clean in some versions. In some instances, it is desirable to confirm the pressure within basin (14a) and within indicator module (512) are comparable to have high confidence in the cleaning correlation provided by indicator module (512) and test coupon (540).

Still in some instances, the pressure within indicator module (512) as determined by sensor (562) is desired to match the pressure not within basin (14a), but instead within the channels of endoscope (200) that is placed within basin (14a). Based on the inputs provided to control system (20) on the conditions within decontamination station (510), a pressure or estimated pressure within the channels of endoscope (200) can be determined or calculated. This can then be compared to the measured pressure at sensor (562). To control the pressure within indicator module (512), either or both of air from air supply (516) and fluid from basin (14a) can be directed to or away from indicator module (512) to make pressure changes to match or substantially match the desired conditions being experienced within channels of endoscope (200) within basin (14a).

In another example, indicator module (512) includes a flow rate sensor (564) configured to detect the flow rate of the fluid within indicator module (512). In this manner, the flow rate that sample well (544) is subject to is known or determinable. In addition to temperature, and/or pressure, flow rate can also impact cleaning ability or decontamination progress and time to clean or decontaminate in some versions. In some instances, it is desirable to confirm the flow rate within basin (14a) and within indicator module (512) are comparable to have high confidence in the cleaning or decontamination correlation provided by indicator module (512) and test coupon (540).

Still in some instances, the flow rate within indicator module (512) as determined by sensor (564) is desired to match the flow rate not within basin (14a), but instead within the channels of endoscope (200) that is placed within basin (14a). Based on the inputs provided to control system (20) on the conditions within decontamination station (510), a flow rate or estimated flow rate within the channels of endoscope (200) can be determined or calculated. This can then be compared to the measured flow rate at sensor (564). To control or adjust the flow rate within indicator module (512), pump (530) can be controlled to increase or decrease flow as needed to make flow rate changes to match or substantially match the desired conditions being experienced within channels of endoscope (200) within basin (14a).

In another example, indicator module (512) includes a concentration sensor (566) configured to detect the concentration of the fluid within indicator module (512). For instance, where the fluid is a detergent or a disinfectant of known or desired concentration, concentration sensor (566) can measure the concentration of such fluid within indicator module (512). In this manner, the concentration that sample well (544) is subject to is known or determinable. In addition to temperature, and/or pressure, and/or flow rate, concentration can also impact cleaning ability or decontamination progress and time to clean or decontaminate in some versions. In some instances, it is desirable to confirm the concentration of the fluid within basin (14a) and within indicator module (512) are comparable to have high confidence in the cleaning or decontamination correlation provided by indicator module (512) and test coupon (540).

Still in some instances, the concentration within indicator module (512) as determined by sensor (566) is desired to match the concentration not within basin (14a), but instead within the channels of endoscope (200) that is placed within basin (14a). Based on the inputs provided to control system (20) on the conditions within decontamination station (510), a concentration or estimated concentration within the channels of endoscope (200) can be determined or calculated. This can then be compared to the measured concentration at sensor (566). To control or adjust the concentration within indicator module (512), flush lines (511, 513) from various chemical supply containers can be connected with indicator module (512), in addition to a flush line (515) connecting indicator (512) with water source (50), so a calculated chemical concentration can be delivered to indicator module (512) such that the concentration of the chemical or agent within the fluid within indicator module (512) can match the conditions experienced in basin (14a) or within the channels of endoscope (200) as the case may be.

While the above examples are presented in the context of reprocessing endoscopes, it will be apparent to those of ordinary skill in the art, in view of the teachings herein, that these same or similar techniques and/or devices can be used to decontaminate or sterilize devices other than endoscopes.

### V. EXEMPLARY MEDICAL DEVICE REPROCESSING METHOD WITH INDICATOR MODULE

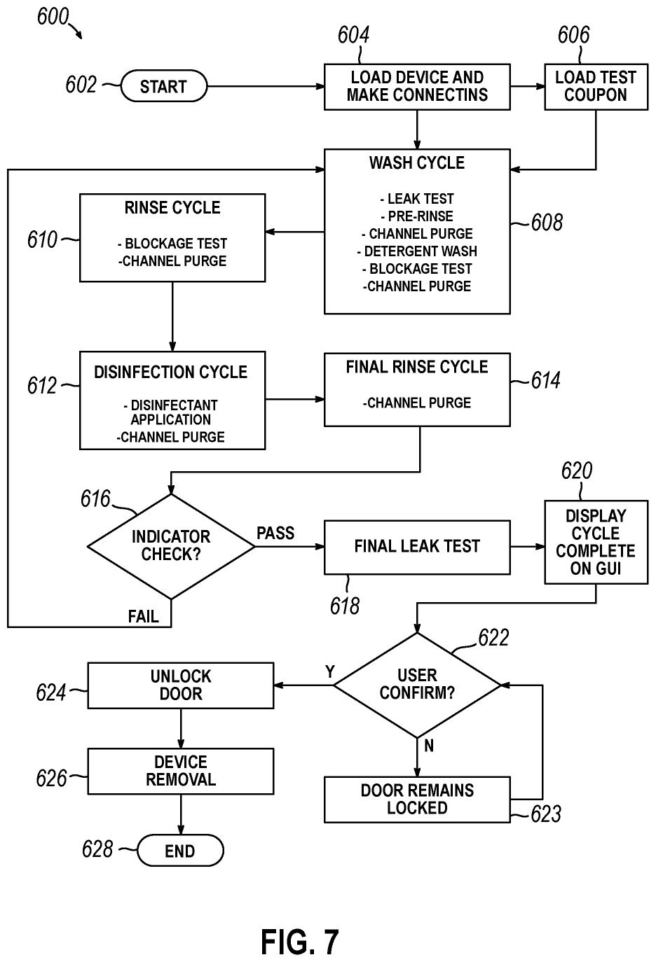

Referring to FIG. 7, in an exemplary method or use (600) of a reprocessing system such as reprocessing system (2) configured with decontamination station (510), an operator may start (602) by loading a device for reprocessing and making any needed connections with the device (604). To do so, the operator may actuate a foot pedal (not shown) to open basin lid (16a). With lid (16a) open, the operator inserts shaft (208) of endoscope (200) into helical circulation tube (64). End section (224) and head section (202) of endoscope (200) are situated within basin (14a), with feed hose (222) coiled within basin (14a) with as wide a diameter as possible. Next, flush lines (30) are attached to respective endoscope openings (226, 228, 228a, 230, 232). Air line (112) is also connected to connector (254). In some versions, flush lines (30) are color coded, and guide located on station (510) provides a reference for the color-coded connections.

Depending on the customer-selectable configuration, control system (20) may prompt the operator to enter a user code, patient ID, endoscope code, and/or specialist code. This information may be entered manually (e.g., through touch screen (22)), automatically (e.g., by using an attached barcode wand), or in any other suitable fashion. With the information entered (if required), the operator may then close lid (16a).

After or prior to loading endoscope (200) and making connections, the operator presses a button on touch-screen (22) to load an indicator such as test coupon (540) within indicator module (512) (606) by activating test coupon loading feature (558). Test coupon (540) may be a cleaning test coupon or may be configured as a chemical or biological indicator. The various types of test coupons (540) usable with decontamination station (510) and method (600) will be apparent to those of ordinary skill in the art in view of the teachings herein.

With endoscope (200) and test coupon (540) loaded, once lid (16a) is closed, the operator presses a button on touch-screen (22) to begin the wash cycle (608). At the start of wash cycle (608), leak testing is performed as explained above with respect to FIG. 4 and method (400). After leak tests are passed, reprocessing system (2) continues wash cycle (608) with a pre-rinse cycle. The purpose of this step is to flush water through channels (210, 212, 213, 214, 217, 218) to remove waste material prior to washing and disinfecting endoscope (200). To initiate the pre-rinse cycle, basin (14a) is filled with filtered water and the water level is detected by pressure sensor (59) below basin (14a). The water is pumped via pumps (32) through the interior of channels (210, 212, 213, 214, 217, 218), directly to drain (74). This water is not recirculated around the exterior surfaces of endoscope (200) during this stage. As the water is being pumped through channels (210, 212, 213, 214, 217, 218), drain pump (72) is activated to ensure that basin (14a) is also emptied. Drain pump (72) will be turned off when drain switch (76) detects that the drain process is complete. During the draining process, a channel purge is executed where sterile air is blown via air pump (38) through all endoscope channels (210, 212, 213, 214, 217, 218) simultaneously, to minimize potential carryover.

In the present example, test coupon (540) is subjected to the same conditions as the internal channels of endoscope (200). Therefore, a pre-rinse cycle also occurs within indicator module (512) contemporaneously with the pre-rinse cycle within basin (14a). Accordingly, after basin (14a) is filled with water, water from basin (14a) is transferred to indicator module (512) by actuating valves (532, 534, 536) and pump (530). This water flows over and past test coupon (540) and its reference well (542) and sample well (544). The water flows to drain (74) after flowing past test coupon (504). In some other versions subjecting test coupon (540) to the pre-rinse cycle is optional.

Once the pre-rinse cycle is complete, wash cycle (608) continues by filling basin (14a) with warm water (e.g., approximately 35° C.). Water temperature is controlled by controlling the mix of heated and unheated water. The water level is detected by pressure sensor (59). Reprocessing system (2) then adds enzymatic detergent from detergent solution (86) to the water circulating in reprocessing system (2) by means of peristaltic metering pump (88). The volume is controlled by controlling the delivery time, pump speed, and inner diameter of the tubing of pump (88). At this point valves (532, 534, 536) and pump (530) are activated to transfer fluid from basin (14a) into indicator module (512). The fluid comprised of water and detergent solution (86) is actively pumped throughout the internal endoscope channels (210, 212, 213, 214, 217, 218) and over the outer surface of endoscope (200) for a predetermined time period (e.g., from one to five minutes, or more particularly about three minutes), by channel pumps (32) and external circulation pump (70). Inline heater (80) keeps the temperature at a predetermined temperature (e.g., approximately about 35° C.).

Contemporaneously, this fluid comprised of water and detergent solution (86) is pumped past test coupon (540) and its reference and sample wells (542, 544). This continues for the same predetermined time period that this fluid is actively pumped throughout the internal endoscope channels (210, 212, 213, 214, 217, 218). During this process, control system (20) controls temperature control feature (528) based on input temperature information from probe (522) and sensor (560) to maintain the temperature of the fluid within indicator module (512) to match or mirror the temperature of the fluid comprised of water and detergent solution (86) in basin (14a) that is being circulated through the endoscope channels.

After circulating for a certain period of time (e.g., a couple of minutes), the flow rate through channels (210, 212, 213, 214, 217, 218) is measured. If the flow rate through any channel (210, 212, 213, 214, 217, 218) is less than a predetermined rate for that channel (210, 212, 213, 214, 217, 218), the channel (210, 212, 213, 214, 217, 218) is identified as blocked, the program is stopped, and the operator is notified of the condition. Peristaltic pumps (32) are run at their predetermined flow rates and cycle off in the presence of unacceptably high pressure readings at the associated pressure sensor (42). If a channel (210, 212, 213, 214, 217, 218) is blocked, the predetermined flow rate will trigger pressure sensor (42), indicating the inability to adequately pass this flow rate. As pumps (32) are peristaltic in the present example, their operating flow rate combined with the percentage of time they are cycled off due to pressure will provide the actual flow rate. The flow rate can also be estimated based upon the decay of the pressure from the time pump (32) cycles off.

At the end of wash cycle (608), drain pump (72) is activated to remove the fluid from basin (14a) and channels (210, 212, 213, 214, 217, 218). Drain pump (72) turns off when drain level sensor (76) indicates that drainage is complete. Similarly, pump (530) directs fluid from indicator module (512) to drain (74). During the drain process, sterile air is blown through all channels (210, 212, 213, 214, 217, 218) of endoscope (200) simultaneously to purge the channels and minimize potential carryover.

In some other versions of wash cycle (608), instead of channels (210, 212, 213, 214, 217, 218) of endoscope (200) being filled with the same fluid within basin (14a), they are filled with either undiluted detergent solution (86) or a fluid comprised of water and detergent solution (86) having a different concentration than that within basin (14a), e.g. a higher concentration. In such a version, the undiluted detergent solution (86) or the fluid having the different concentration from that of the fluid within basin (14a) is directed to indicator module (512) to expose test coupon (540) to the same fluid that is being passed through channels (210, 212, 213, 214, 217, 218) of endoscope (200).

Next, reprocessing system (2) begins a rinse cycle (610). To initiate rinse cycle (610), basin (14a) is again filled with warm water (e.g., at approximately 35° C.). Water temperature is controlled by controlling the mix of heated and unheated water. The water level is detected by pressure sensor (59). The rinse water is circulated within channels (210, 212, 213, 214, 217, 218) of endoscope (200) via channel pumps (32); and over the exterior of endoscope (200) via circulation pump (70) and sprinkler arm (60) for a certain period of time (e.g., one minute). As rinse water is pumped through channels (210, 212, 213, 214, 217, 218), the flow rate through channels (210, 212, 213, 214, 217, 218) is measured and if it falls below the predetermined rate for any given channel (210, 212, 213, 214, 217, 218), that channel (210, 212, 213, 214, 217, 218) is identified as blocked, the program is stopped, and the operator is notified of the condition.

Contemporaneously, warm water from basin (14a) is pumped to indicator module (512) and past test coupon (540) and its reference and sample wells (542, 544). This continues for the same predetermined time period that rinse water is actively pumped throughout the internal endoscope channels (210, 212, 213, 214, 217, 218). During this process, control system (20) controls temperature control feature (528) based on input temperature information from probe (522) and sensor (560) to maintain the temperature of the fluid within indicator module (512) to match or mirror the temperature of the rinse water in basin (14a) that is being circulated through the endoscope channels and around the endoscope exterior.

At the end of rinse cycle (610), drain pump (72) is activated to remove the rinse water from basin (14a) and channels (210, 212, 213, 214, 217, 218). Drain pump (72) turns off when drain level sensor (76) indicates that drainage is complete. Similarly, pump (530) directs fluid from indicator module (512) to drain (74). During the drain process, sterile air is blown through all channels (210, 212, 213, 214, 217, 218) of endoscope (200) simultaneously to purge the channels and minimize potential carryover. In some versions, the above-described rinsing and draining cycles are repeated at least once again, to ensure maximum rinsing of detergent solution (86) from the surfaces of endoscope (200), basin (14a), and indicator module (512).

Next, reprocessing system (2) proceeds to a disinfection cycle (612). To initiate disinfection cycle (612), basin (14a) is filled with very warm water (e.g., at approximately 53° C.). Water temperature is controlled by controlling the mix of heated and unheated water. The water level is detected by pressure sensor (59). During the filling process, channel pumps (32) are off in order to ensure that the disinfectant solution (92) in basin (14a) is at the in-use concentration prior to circulating through channels (210, 212, 213, 214, 217, 218) of endoscope (200).

Next, a measured volume of disinfectant solution (92) is drawn from disinfectant metering pre-chamber (96) and delivered into the water in basin (14a) via metering pump (100). The volume of disinfectant solution (92) is controlled by the positioning of fill level switch (98) relative to the bottom of metering pre-chamber (96). Metering pre-chamber (96) is filled until fill level switch (98) detects liquid. Disinfectant solution (92) is drawn from metering pre-chamber (96) until the level of disinfectant solution (92) in metering pre-chamber (96) is just below the tip of metering pre-chamber (96). After the necessary volume is dispensed, metering pre-chamber (96) is refilled from the bottle of disinfectant solution (92). Disinfectant solution (92) is not added until basin (14a) is filled, so that in case of a water supply problem, concentrated disinfectant is not left on endoscope (200) with no water to rinse it. While disinfectant solution (92) is being added, channel pumps (32) are off in order to ensure that disinfectant solution (92) in basin (14a) is at the desired in-use concentration prior to circulating through channels (210, 212, 213, 214, 217, 218) of endoscope (200).

The fluid at the in-use concentration comprised of water and disinfectant solution (92) is actively pumped throughout internal channels (210, 212, 213, 214, 217, 218) by pumps (32) and over the outer surface of endoscope (200) by circulation pump (70). This may be done for any suitable duration (e.g., at least 5 minutes). The temperature of the fluid comprised of water and disinfectant solution (92) may be controlled by in-line heater (80) to stay at a consistent temperature (e.g., about 52.5° C.). During the disinfection process, flow through each channel (210, 212, 213, 214, 217, 218) of endoscope (200) is verified by timing the delivery of a measured quantity of solution through channel (210, 212, 213, 214, 217, 218). Valve (S1) is closed, and valve (S7) opened, and in turn each channel pump (32) delivers a predetermined volume to its associated channel (210, 212, 213, 214, 217, 218) from metering tube (136). This volume and the time it takes to deliver the volume, provides a very accurate flow rate through the channel (210, 212, 213, 214, 217, 218). Anomalies in the flow rate from what is expected for a channel (210, 212, 213, 214, 217, 218) of that diameter and length are flagged by control system (20) and the process stopped. As the fluid comprised of water and disinfectant solution (92) is pumped through channels (210, 212, 213, 214, 217, 218), the flow rate through channels (210, 212, 213, 214, 217, 218) is also measured as described above.

Contemporaneously, the fluid comprised of water and disinfectant solution (92) from basin (14a) is pumped to indicator module (512) and past test coupon (540) and its reference and sample wells (542, 544). This continues for the same predetermined time period that this fluid is actively pumped throughout the internal endoscope channels (210, 212, 213, 214, 217, 218). During this process, control system (20) controls temperature control feature (528) based on input temperature information from probe (522) and sensor (560) to maintain the temperature of the fluid within indicator module (512) to match or mirror the temperature of the fluid comprised of water and disinfectant solution (92) in basin (14a) that is being circulated through the endoscope channels and around the endoscope exterior.

At the end of disinfection cycle (612), drain pump (72) is activated to remove the fluid from basin (14a) and channels (210, 212, 213, 214, 217, 218). Similarly, pump (530) directs fluid from indicator module (512) to drain (74). During the draining process, sterile air is blown through all channels (210, 212, 213, 214, 217, 218) of endoscope (200) simultaneously to purge channels and minimize potential carryover.

In some other versions of disinfection cycle (612), instead of channels (210, 212, 213, 214, 217, 218) of endoscope (200) being filled with the same fluid within basin (14a), they are filled with either undiluted disinfectant solution (92) or a fluid comprised of water and disinfectant solution (92) having a different concentration than that within basin (14a), e.g. a higher concentration. In such a version, the undiluted disinfectant solution (92) or the fluid having the different concentration from that of the fluid within basin (14a) is directed to indicator module (512) to expose test coupon (540) to the same fluid that is being passed through channels (210, 212, 213, 214, 217, 218) of endoscope (200).

Next, reprocessing system (2) begins a final rinse cycle (614). To initiate cycle (614), basin (14a) is filled with sterile warm water (e.g., at approximately 45° C.) that has been passed through a filter (e.g., a 0.2 μm filter). The rinse water is circulated within channels (210, 212, 213, 214, 217, 218) by pumps (32); and over the exterior of endoscope (200) via circulation pump (70) and sprinkler arm 60) for a suitable duration (e.g., 1 minute). As rinse water is pumped through channels (210, 212, 213, 214, 217, 218), the flow rate through channels (210, 212, 213, 214, 217, 218) is measured as described above.

Contemporaneously, warm water from basin (14a) is pumped to indicator module (512) and past test coupon (540) and its reference and sample wells (542, 544). This continues for the same predetermined time period that rinse water is actively pumped throughout the internal endoscope channels (210, 212, 213, 214, 217, 218). During this process, control system (20) controls temperature control feature (528) based on input temperature information from probe (522) and sensor (560) to maintain the temperature of the fluid within indicator module (512) to match or mirror the temperature of the rinse water in basin (14a) that is being circulated through the endoscope channels and around the endoscope exterior.

At the conclusion of final rinse cycle (614), drain pump (72) is activated to remove the rinse water from basin (14a) and channels (210, 212, 213, 214, 217, 218). Similarly, pump (530) directs fluid from indicator module (512) to drain (74). During the draining process, sterile air is blown through all channels (210, 212, 213, 214, 217, 218) of endoscope (200) simultaneously to purge channels and minimize potential carryover. In some versions, the above-described rinsing and draining cycles are repeated at least two more times, to ensure maximum rinsing of disinfectant solution (92) residuals from the surfaces of endoscope (200), basin (14a), and indicator module (512).

Next, reprocessing system (2) begins an indicator check (616). With indicator check (616), readings are taken based on the light transmission detection at reference light detector (548) and sample light detector (552). These are compared to make a determination if the light transmission for sample well (544) is within a predetermined threshold of the light transmission for reference well (542). If within the threshold, then the transmission of light detected is similar such that test coupon (540) now shows that decontamination has occurred to an acceptable or target level. If not within the threshold, then the transmission of light detected is dissimilar such that test coupon (540) indicates that decontamination of sample well (544) does not yet resemble reference well (542), which represents an acceptable standard or amount of decontamination. Therefore, further wash, disinfect, and rinse cycles are performed as shown in FIG. 7.

Once indicator check (616) passes based on the light transmission detection as noted above, reprocessing system (2) begins a final leak test (618). In particular, reprocessing system (2) pressurizes the body of endoscope (200) and measures the leak rate as described above. If the final leak test is successful, reprocessing system (2) indicates the successful completion of the cycles by displaying a cycle complete indication (620) on a graphical user interface (GUI), e.g., via touch-screen (22). From the time of program completion to the time at which lid (16a) is opened, pressure within the body of endoscope (200) is normalized to atmospheric pressure by opening vent valve (S5) at a predetermined rate (e.g., valve (S5) opened for 10 seconds every minute).

Depending on customer-selected configuration, reprocessing system (2) may prevent lid (16a) from being opened, where the door remains locked (623) until receiving user confirmation (622), e.g., a valid user identification code may be entered to provide user confirmation. Information about the completed program, including the user ID, endoscope ID, specialist ID, and patient ID are stored along with the sensor data obtained throughout the program. If a printer is connected to reprocessing system (2), and if requested by the operator, a record of the disinfection program will be printed. Once user confirmation (622) has been received, lid (16a) may be unlocked (624) and opened (e.g., using the foot pedal as described above). Endoscope (200) is then removed in a device removal step (626) where endoscope (200) is disconnected from flush lines (30) and removed from basin (14a). Lid (16a) can then be closed using both the hardware and software buttons as described above. With device (200) removed from reprocessing system (2), reprocessing method (600) ends (628) and may be repeated thereafter for reprocessing additional devices, i.e. endoscopes (200).

In some versions of method (600), indicator check (616) may occur multiple times through method (600). For instance, an indicator check (616) could be performed after each cycle to monitor the progress of decontamination. In view of the teachings herein, other ways to modify method (600) will be apparent to those of ordinary skill in the art.

### VI. EXEMPLARY COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus for reprocessing a device comprises (a) a basin configured to receive the device for reprocessing, wherein the basin is further configured to receive one or more fluids to aid in reprocessing; (b) one or more pumps configured to transfer the one or more fluids to and from the basin; (c) one or more valves configured to direct the one or more fluids being transferred by the one or more pumps; and (d) a module fluidly connected with, but physically separate from, the basin, wherein the module is configured to receive one or more indicators, wherein the one or more indicators are configured to signal when a predetermined level of decontamination has been achieved.

### Example 2

The apparatus of Example 1, wherein a first condition exists within the basin, and wherein a second condition exists within the module, wherein the first condition is measured or calculated, and wherein the second condition is controlled based on the measured or calculated first condition such that the second condition within the module substantially matches the first condition within the basin.

### Example 3

The apparatus of any one or more of Example 1 through Example 2, wherein the one or more fluids received by the basin are transferred to the module such that the basin and the module contain the same fluid.

### Example 4

The apparatus of any one or more of Example 1 through Example 3, comprising a first temperature sensor located within the basin and configured to measure a first temperature of a select one of the one or more fluids within the basin.

### Example 5

The apparatus of Example 4, comprising a material surrounding the first temperature sensor, wherein the material is configured to provide insulating properties that resemble insulating properties the device provides to an internal region of the device such that the first temperature measured by the first temperature sensor is representative of temperature within the internal region of the device.

### Example 6

The apparatus of any one or more of Example 1 through Example 5, comprising a temperature control feature configured to provide heat or cooling to the one or more fluids received by the module.

### Example 7

The apparatus of Example 6, wherein the temperature control feature is controlled based upon the first temperature measured by the first temperature sensor located within the basin.

### Example 8

The apparatus of any one or more of Example 1 through Example 7, comprising a second temperature sensor located within the module, wherein the first temperature and the second temperature are provided as inputs to a controller and define a temperature feedback loop, wherein the controller controls the temperature control feature based upon the temperature feedback loop.

### Example 9

The apparatus of any one or more of Example 1 through Example 8, wherein the one or more indicators comprise a reference well and a sample well, wherein the module comprises a reference light source, a reference light detector, a sample light source, and a sample light source detector, wherein the reference light detector detects light transmitted from the reference light source through the reference well, and wherein the sample light detector detects light transmitted from the sample light source through the sample well.

### Example 10

The apparatus of Example 9, wherein a successful decontamination state exists when the light transmission detected at the reference light detector and the sample light detector is substantially equal.

### Example 11

The apparatus of any one or more of Example 1 through Example 10, wherein the module comprises an indicator loading feature configured to position the one or more indicators within the module.

### Example 12

The apparatus of any one or more of Example 1 through Example 11, comprising a select one or more of a pressure sensor, a concentration sensor, and a flowrate sensor positionable within the module to provide respective pressure, concentration, and flowrate data.

### Example 13

The apparatus of Example 12, wherein a select one or more of a first pressure within the module, a first concentration within the module, and a first flowrate of the one or more fluids within the module are adjusted to substantially match a second pressure within the basin or within one or more channels of the device, a second concentration within the basin or within the one or more channels of the device, and a second flowrate of the one or more fluids within the basin or within the one or more channels of the device.

### Example 14

The apparatus of any one or more of Example 1 through Example 13, comprising one or more flush lines, wherein the device comprises one or more internal channels, wherein the one or more flush lines are configured to connect with a respective one of the one or more internal channels of the device to direct the one or more fluids to the one or more internal channels of the device.

### Example 15

The apparatus of Example 14, wherein one or more conditions within the module are configured to substantially match respective conditions within the one or more internal channels of the device positioned within the basin.

### Example 16

A method of reprocessing a device having one or more channels using a reprocessing apparatus, comprises: (a) loading the device having the one or more channels into a basin of the reprocessing apparatus; (b) connecting each of the one or more channels of the device with one or more flush lines; (c) transferring one or more fluids to the basin of the reprocessing apparatus; (d) transferring the one or more fluids transferred to the basin to a module of the reprocessing apparatus that is physically separate from the basin of the reprocessing system such that the module is located remote from the device within the basin, wherein the module comprises one or more indicators configured to communicate a decontamination status; (e) controlling one or more parameters of the one or more fluids transferred to the basin and the module so that the one or more parameters present within the basin and within the module substantially match; and (f) evaluating the one or more indicators within the module to determine the decontamination status, wherein the decontamination status is representative of a level of decontamination of the device within the basin.

### Example 17

The method of Example 16, wherein the one or more parameters is selected from the group consisting of a temperature, a pressure, a flowrate, or combinations thereof.

### Example 18

The method of any one or more of Example 16 through Example 17, wherein the one or more fluids is selected from the group consisting of water, a detergent solution, a disinfectant solution, or combinations thereof.

### Example 19

The method of any one or more of Example 16 through Example 18, wherein evaluating the one or more indicators comprises detecting a reference light transmission passed through a reference well of the one or more indicators, detecting a sample light transmission passed through a sample well of the one or more indicators, and comparing the sample light transmission with the reference light transmission.

### Example 20

The method of any one or more of Example 16 through Example 19, wherein controlling one or more parameters of the one or more fluids transferred to the basin and the module so that the one or more parameters present within the basin and within the module substantially match comprises substantially matching the one or more parameters from within the one or more channels of the device with the one or more parameters within the module containing the one or more indicators.

### Example 21

A method of reprocessing a device having one or more channels using a reprocessing apparatus comprises: (a) loading the device having the one or more channels into a basin of the reprocessing apparatus; (b) connecting each of the one or more channels of the device with one or more flush lines; (c) transferring a fluid to the basin of the reprocessing apparatus, wherein the fluid is directed through the one or more channels of the device; (d) transferring the fluid to a module of the reprocessing apparatus that is physically separate from the basin of the reprocessing system such that the module is located remote from the device within the basin, wherein the module comprises an indicator configured to communicate a decontamination status; (e) controlling one or more parameters of the fluid transferred to the basin and the module, so that the one or more parameters of the fluid present within the one or more channels of the device and within the module having the indicator, substantially match; (f) evaluating the indicator within the module to determine the decontamination status, wherein the decontamination status is representative of a level of decontamination of the one or more channels of the device within the basin; and (g) displaying a cycle complete indication on a graphical user interface of the reprocessing system after achieving a decontamination status that indicates the one or more channels of the device have been decontaminated.

### VII. MISCELLANEOUS

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings. It should be understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

1. An apparatus for reprocessing a device comprising:
(a) a basin configured to receive the device for reprocessing, wherein the basin is further configured to receive one or more fluids to aid in reprocessing;
(b) one or more pumps configured to transfer the one or more fluids to and from the basin;
(c) one or more valves configured to direct the one or more fluids being transferred by the one or more pumps; and
(d) a module fluidly connected with, but physically separate from, the basin, wherein the module is configured to receive one or more indicators, wherein the one or more indicators are configured to signal when a predetermined level of decontamination has been achieved.

2. The apparatus of claim 1, wherein a first condition exists within the basin, and wherein a second condition exists within the module, wherein the first condition is measured or calculated, and wherein the second condition is controlled based on the measured or calculated first condition such that the second condition within the module substantially matches the first condition within the basin.

3. The apparatus of claim 1, wherein the one or more fluids received by the basin are transferred to the module such that the basin and the module contain the same fluid.

4. The apparatus of claim 1, comprising a first temperature sensor located within the basin and configured to measure a first temperature of a select one of the one or more fluids within the basin.

5. The apparatus of claim 4, comprising a material surrounding the first temperature sensor, wherein the material is configured to provide insulating properties that resemble insulating properties the device provides to an internal region of the device such that the first temperature measured by the first temperature sensor is representative of temperature within the internal region of the device.

6. The apparatus of claim 1, comprising a temperature control feature configured to provide heat or cooling to the one or more fluids received by the module.

7. The apparatus of claim 6, wherein the temperature control feature is controlled based upon the first temperature measured by the first temperature sensor located within the basin.

8. The apparatus of claim 1, comprising a second temperature sensor located within the module, wherein the first temperature and the second temperature are provided as inputs to a controller and define a temperature feedback loop, wherein the controller controls the temperature control feature based upon the temperature feedback loop.

9. The apparatus of claim 1, wherein the one or more indicators comprise a reference well and a sample well, wherein the module comprises a reference light source, a reference light detector, a sample light source, and a sample light source detector, wherein the reference light detector detects light transmitted from the reference light source through the reference well, and wherein the sample light detector detects light transmitted from the sample light source through the sample well.

10. The apparatus of claim 9, wherein a successful decontamination state exists when the light transmission detected at the reference light detector and the sample light detector is substantially equal.

11. The apparatus of claim 1, wherein the module comprises an indicator loading feature configured to position the one or more indicators within the module.

12. The apparatus of claim 1, comprising a select one or more of a pressure sensor, a concentration sensor, and a flowrate sensor positionable within the module to provide respective pressure, concentration, and flowrate data.

13. The apparatus of claim 12, wherein a select one or more of a first pressure within the module, a first concentration within the module, and a first flowrate of the one or more fluids within the module are adjusted to substantially match a second pressure within the basin or within one or more channels of the device, a second concentration within the basin or within the one or more channels of the device, and a second flowrate of the one or more fluids within the basin or within the one or more channels of the device.

14. The apparatus of claim 1, comprising one or more flush lines, wherein the device comprises one or more internal channels, wherein the one or more flush lines are configured to connect with a respective one of the one or more internal channels of the device to direct the one or more fluids to the one or more internal channels of the device, wherein one or more conditions within the module are configured to substantially match respective conditions within the one or more internal channels of the device positioned within the basin.

15. (canceled)

16. A method of reprocessing a device having one or more channels using a reprocessing apparatus, the method comprising:
(a) loading the device having the one or more channels into a basin of the reprocessing apparatus;
(b) connecting each of the one or more channels of the device with one or more flush lines;
(c) transferring one or more fluids to the basin of the reprocessing apparatus;
(d) transferring the one or more fluids transferred to the basin to a module of the reprocessing apparatus that is physically separate from the basin of the reprocessing system such that the module is located remote from the device within the basin, wherein the module comprises one or more indicators configured to communicate a decontamination status;
(e) controlling one or more parameters of the one or more fluids transferred to the basin and the module so that the one or more parameters present within the basin and within the module substantially match; and
(f) evaluating the one or more indicators within the module to determine the decontamination status, wherein the decontamination status is representative of a level of decontamination of the device within the basin.

17. The method of claim 16, wherein the one or more parameters is selected from the group consisting of a temperature, a pressure, a concentration, a flowrate, or combinations thereof.

18. The method of claim 16, wherein the one or more fluids is selected from the group consisting of water, a detergent solution, a disinfectant solution, or combinations thereof.

19. The method of claim 16, wherein evaluating the one or more indicators comprises detecting a reference light transmission passed through a reference well of the one or more indicators, detecting a sample light transmission passed through a sample well of the one or more indicators, and comparing the sample light transmission with the reference light transmission.

20. The method of claim 16, wherein controlling one or more parameters of the one or more fluids transferred to the basin and the module so that the one or more parameters present within the basin and within the module substantially match comprises substantially matching the one or more parameters from within the one or more channels of the device with the one or more parameters within the module containing the one or more indicators.

21. A method of reprocessing a device having one or more channels using a reprocessing apparatus, the method comprising:
(a) loading the device having the one or more channels into a basin of the reprocessing apparatus;
(b) connecting each of the one or more channels of the device with one or more flush lines;
(c) transferring a fluid to the basin of the reprocessing apparatus, wherein the fluid is directed through the one or more channels of the device;
(d) transferring the fluid to a module of the reprocessing apparatus that is physically separate from the basin of the reprocessing system such that the module is located remote from the device within the basin, wherein the module comprises an indicator configured to communicate a decontamination status;
(e) controlling one or more parameters of the fluid transferred to the basin and the module, so that the one or more parameters of the fluid present within the one or more channels of the device and within the module having the indicator, substantially match;
(f) evaluating the indicator within the module to determine the decontamination status, wherein the decontamination status is representative of a level of decontamination of the one or more channels of the device within the basin; and
(g) displaying a cycle complete indication on a graphical user interface of the reprocessing system after achieving a decontamination status that indicates the one or more channels of the device have been decontaminated.
